(19) United States
(12) Patent Application Publication
(10) Pub. No.: US 2013/0267860 A1
(43) Pub. Date: Oct. 10, 2013

(54) SEED-BEAT SELECTION METHOD FOR SIGNAL-AVERAGED ELECTROCARDIOGRAPHY

(71) Applicant: Arrhythmia Research Technology, Inc., Fitchburg, MA (US)

(72) Inventors: Eric K. Y. Chan, San Carlos, CA (US); Brian D. Faldasz, Montague, MA (US)

(21) Appl. No.: 13/800,207

(22) Filed: Mar. 13, 2013

Related U.S. Application Data
(60) Provisional application No. 61/685,170, filed on Mar. 13, 2012.

(51) Int. Cl. A61B 5/04

(52) U.S. Cl. CPC A61B 5/04012
USPC 600/509

(57) ABSTRACT

An improved method for selection of a seed beat for performing signal averaging on electrocardiograms (“ECGs”). The invention improves upon the prior art by extending the search for seed beats further into the electrocardiogram time-series and by searching all three orthogonal electrocardiographic channels. The method successfully locates seed beats even in suboptimal ECGs dramatically improving the success rate for analyzing ECGs eliminating the need to re-perform ECG analysis thus improving efficiency and reducing delay in discovery of dangerous arrhythmias.

### RELATED APPLICATION

This application claims priority from Applicants' U.S. Provisional Patent Application, Ser. No. 61/685,170, filed Mar. 13, 2012, entitled “Method of Seed-Beat Selection for Signal-Averaged Electrocardiography.” Applicants hereby claim the benefit of priority from that provisional application. Applicants also hereby incorporate by reference the entire disclosure from that earlier application.

### FIELD OF INVENTION

This invention relates generally to analysis of electrocardiograms. More particularly, it relates to signal-averaged electrocardiography (“SAECG”).

### BACKGROUND OF THE INVENTION

Through the course of a human lifetime, the human heart beats approximately two billion times. A normal heart beats in a regular, repeating, periodic and predictable way. An electrical pulse that originates in the heart's sinoatrial (“SA”) node—the heart's “pacemaker”—tissue in the right atrium—initiates each heartbeat. The electrical pulse conducts along nerves and from one cell to the next throughout the heart muscle. That pulse causes the muscle to contract in such a way as to force blood into and through the chambers of the heart and out into the circulatory system. The electrical activity that occurs in the heart during each heartbeat gives rise to electrical potentials measurable at the skin surface. By measuring electrical potentials at different locations on the skin, it is possible to determine whether the heart muscle is behaving normally, and if not, then it is further possible to determine what may be causing the abnormality, the location of any pathology, and whether and how likely an abnormality could lead to sudden death.

Analyzing heartbeats using signal-averaged electrocardiography (“SAECG”) relies on the fact that the normal heart beats in a regular periodic manner known as a sinus rhythm.

Electrical activity in the heart generates waveforms that correspond to the transmission of electrical signals through heart tissue during each heartbeat. (Generally, see Applicants' FIGS. 1 and 2). For ease of analysis, waveforms often are converted from analog form into a high-resolution (“HI-RES”) digital format that can be stored and manipulated by computer.

Since the heartbeat is so regular, it is possible to average the shape (or morphology) of a series of heartbeat waveforms to obtain an “average” waveform and then to closely inspect the resulting waveform for anomalies. Signal averaging a series of heartbeats removes some of the interferences and small errors introduced in the measurement of individual beats. SAECG also enhances, with the aid of complex mathematics, the detection of micro-potentials that appear on the averaged heartbeat waveform. Damaged heart tissue does not participate equally with normal heart tissue in the propagation and conduction of electricity through the heart. When damaged heart tissue interferes in the conduction of electricity, it causes a fraction of the potential to arrive late at the surface of the skin. Small, late-arriving potentials (see, e.g., Applicants' FIGS. 4, 4A) may be the harbinger of sudden cardiac death because in some cases they are strongly associated with a propensity for ventricular tachycardia—a deadly form of arrhythmia.

Averaging of heartbeat waveforms requires that the beats be relatively uniform. There are a number of factors that cause difficulty in the measurement and averaging of surface potentials. These include signal noise, contact disruption, baseline drift, and other changes in the signal path caused by patient movement, breathing, and the like. To obtain satisfactory results, signal averaging must start on a heartbeat followed by several similar well-formed heartbeats. The starting beat is commonly called the “seed beat”.

Selecting a seed beat is typically done by a medical practitioner visually observing the displayed electrocardiogram and looking for a series of well-formed beats. However with a suitably sophisticated method, the process can be automatically accomplished by machine.

SAECG data acquisition requires a stable signal environment for the auto-templating process to successfully identify candidate beats. “Suboptimal” high resolution electrocardiograms (“HI-RES ECG”) are characterized by the presence of noisy signals or intermittently changing ventricular systole (“QRS”) morphologies, along with baseline drift due to respiratory artifacts and patient movement. The success rate of automatic template formation in the prior art (e.g., U.S. Pat. No. 4,422,459 to Simson and U.S. Pat. No. 5,025,794 to Albert et al.) and hence yield for completing a SAECG study is greatly reduced in such suboptimal data files, and patients have to be recalled for repeat data acquisition.

In the prior art, automated analysis of SAECG data has been confounded by arbitrary selection of seed beats only within the first several seconds of data collection. For example, PREDICTOR® software by Arrhythmia Research Technology, Inc., bypasses the first eight beats in the data-set and then checks for stability only once and only on a fixed series or set of four heartbeats. If the analysis begins in a region of the electrocardiogram that is not well suited to SAECG—a “suboptimal” electrocardiogram, then there is a high incidence of failure. In such cases, the opportunity to predict deadly arrhythmias is delayed and the patient may need to be re-called to collect improved data.

Accordingly, it is the primary object of this invention to provide an improved method of selecting seed beats for signal averaging of electrocardiograms.

It is another object that this method will enable automated analysis even of suboptimal electrocardiograms, whether freshly collected or previously collected, that would otherwise be unsuitable for automatic analysis by systems using previously available methods.

### SUMMARY OF THE INVENTION

Applicants have disclosed an improved method to locate and select within an electrocardiogram (“ECG”) a suitable heartbeat waveform to serve as a “seed beat” for signal-averaged electrocardiography (“SAECG”).

Signal-averaged electrocardiography, used in the risk stratification of patients at risk for sudden cardiac death due to re-entrant ventricular tachycardia, applies ensemble averaging of high resolution ECG complexes during sinus rhythm to detect microvolt signals called ventricular late potentials (“LP”).

Applicants' invention addresses an unmet need for robust signal processing techniques to successfully process challenging suboptimal high resolution electrocardiograms (“HI-RES ECG”) to improve overall yield of SAECG results.

In a broad sense, Applicants' preferred method comprises: automatically (via software) comparing selected sets of four consecutive heartbeats, of a person, located along preordained positions in the X, Y, and Z channels of an orthogonal high resolution digital electrocardiogram to determine whether a particular set of four consecutive heartbeats contains correlated or sufficiently similar heartbeats (i.e., within 99% of each other); and upon finding a set of sufficiently similar heartbeats, identifying heartbeat three or four from that set as a seed beat to perform SAECG analysis. If the preferred method fails to find at least three out of four sufficiently similar heartbeats in any set of four analyzed heartbeats, it searches for other sets of later occurring heartbeats within the ECG having three or four matching beats.

### BRIEF DESCRIPTION OF DRAWINGS

The above and other objects and advantages of the present invention will become more readily apparent upon reading the following description and reviewing the attached drawings in which:

FIG. 1 depicts a typical normal heartbeat waveform;

FIG. 2 depicts an exemplary electrocardiogram trace with a stable baseline and showing normal sinus rhythm;

FIG. 3 depicts an exemplary orthogonal electrocardiogram waveform having three channels (“X”, “Y”, and “Z”) representing waveforms as sensed at different locations on the skin around the heart and showing an unstable baseline;

FIG. 4 depicts a heartbeat waveform bearing evidence of late micro potentials;

FIG. 4A is an enlarged view of the late micro potentials shown in FIG. 4;

FIG. 5 depicts an exemplary view of a suboptimal heartbeat waveform affected by noise;

FIG. 6 depicts an exemplary view of an electrocardiogram time-series with electrical noise;

FIG. 7 is a flow chart of the preferred method; and

FIG. 8 is a flow chart depicting the sequence of steps applied in determining whether at least three of four heartbeats in a sequence are similar and, if so, assessing whether to select beat 3 or 4 in the sequence as the seed beat.

### DETAILED DESCRIPTION OF THE PREFERRED EMBODIMENT(S)

Applicants have disclosed an improved method of automating the selection of seed beats in the analysis of electrocardiograms. The method employs rule-based artificial intelligence to select optimal “seed beats” for analysis of a signal-averaged electrocardiogram, typically called signal-averaged electrocardiography (“SAECG”).

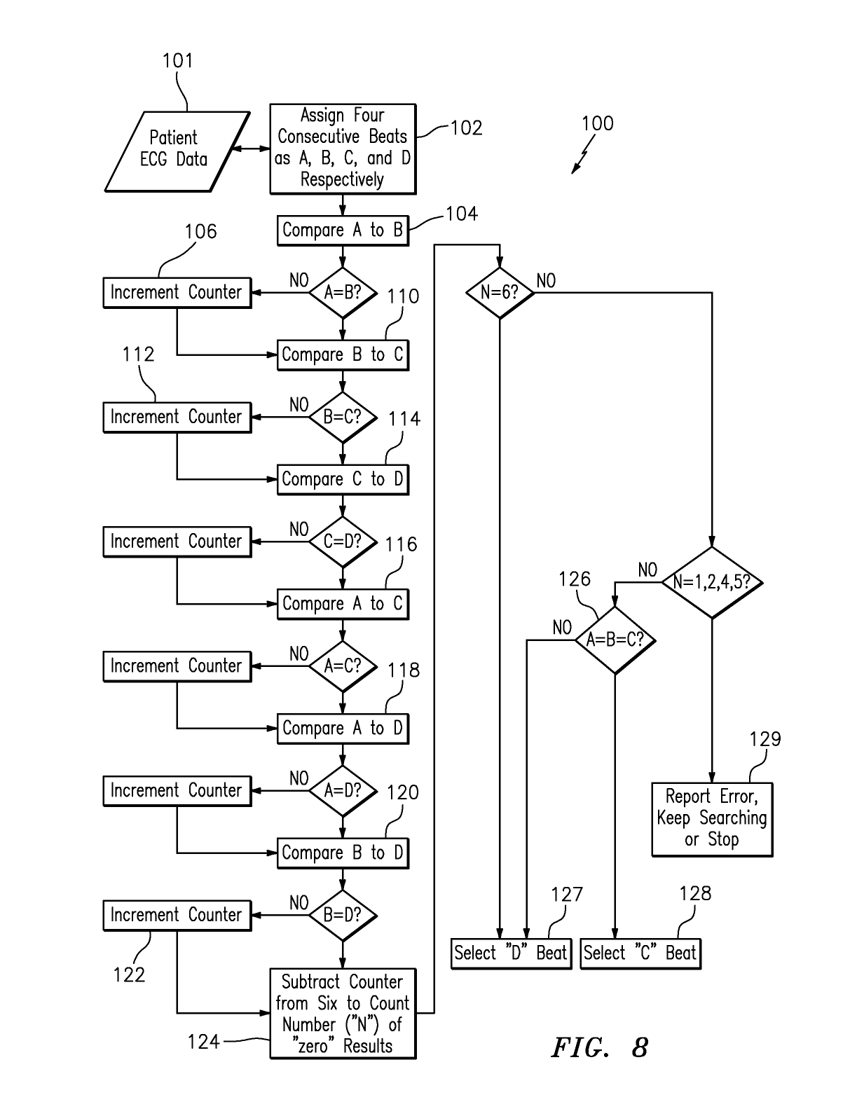

As best shown by FIGS. 7 and 8, Applicants' preferred method 100 comprises: automatically comparing (via software) sets of four consecutive heartbeats located along preordained positions in the X, Y, and Z channels of an orthogonal high resolution digital electrocardiogram to determine whether a particular set of four consecutive heartbeats contains correlated or sufficiently similar heartbeats (i.e., within 99% of each other) to serve as a suitable starting point or “seed beat” for the purposes of performing SAECG analysis; and upon finding a set of sufficiently similar heartbeats, identifying the optimal seed beat from beat three or four of the particular set. If the preferred method fails to find at least three out of four heartbeats in any set of four analyzed heartbeats, it searches for other sets of later occurring heartbeats within the ECG having three or four matching beats. The method inspects waveforms in the X, Y, and Z channels to improve the chances of finding a suitable seed beat. If no candidate seed beat is confirmed, then the method terminates and produces an error message.

Devices that could take advantage of the inventive method with appropriate modification include those described under U.S. Pat. No. 5,609,158, issued Mar. 11, 1997 to Eric K. Y. Chan, and entitled “APPARATUS AND METHOD FOR PREDICTING CARDIAC ARRHYTHMIA BY DETECTION OF MICROPOTENTIALS AND ANALYSIS OF ALL ECG SEGMENTS AND INTERVALS.” Mr. Chan is also a Co-Applicant in the present application. Applicants hereby incorporate the disclosure of that patent by reference.

Briefly, it will be of use to review how an electrocardiogram susceptible of analysis by signal averaging is obtained. First, electrodes are connected to the patient to continuously sense the electrical potentials on the skin surface in three directions orthogonally arranged around the heart and called the “X,” “Y,” and “Z” potentials (often referred to as “leads” or “channels”). The XYZ potentials are measured relative to an electrically neutral or “ground” lead. Every heartbeat produces one heartbeat waveform in each channel. Each of these signals is plotted on a vertical scale against a horizontal time axis (see FIGS. 1-6). The signals are amplified and then digitized by passing the analog signals through an analog-to-digital converter (“ADC”). The ADC samples the analog signal typically 1000 times (or more) per second per channel and stores the data with at least 12 to 16 bits of binary resolution. A computer stores, manipulates, reduces, and displays the resulting electrocardiographic data as it is collected. Data can be stored on a recording device for later analysis by computer or it can be stored directly on a computer and analyzed immediately. The choice of when to analyze the data after collection is not relevant to the present invention except that it is the object of the invention to allow its use either contemporaneously or on previously collected and stored electrocardiograms.

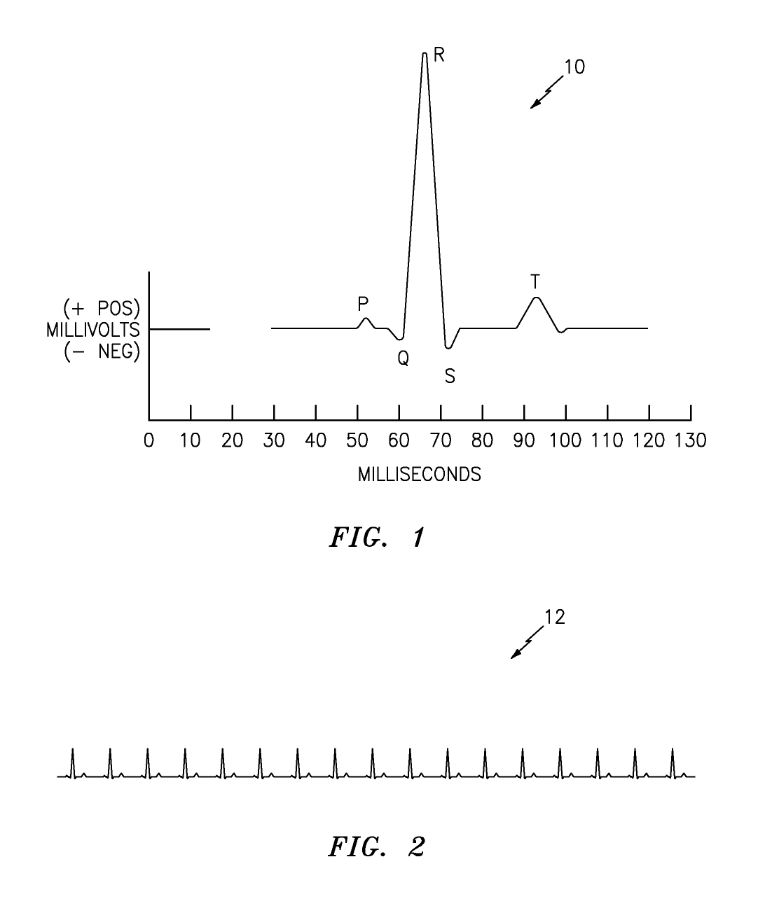

Applicants' FIG. 1 depicts a typical normal heartbeat waveform 10. FIG. 2 depicts an exemplary electrocardiogram trace showing a normal sinus rhythm 12.

The sensing of electrical potentials can be affected by many factors including the quality of the electrical connection at each measurement location on the patient's skin. Other factors include changes in the physical position of the patient's body including the expansion and contraction of the chest wall during breathing, stray electrical potentials caused by static electricity, and induced electrical disturbances from, for example, nearby lighting fixtures, cell phones, and the like. The quality of amplifying, filtering, and digitizing electronics also play a role in the quality of the resulting electrocardiogram. Baseline shifting and noise are two prominent adverse effects that cause difficulty in electronic signal averaging.

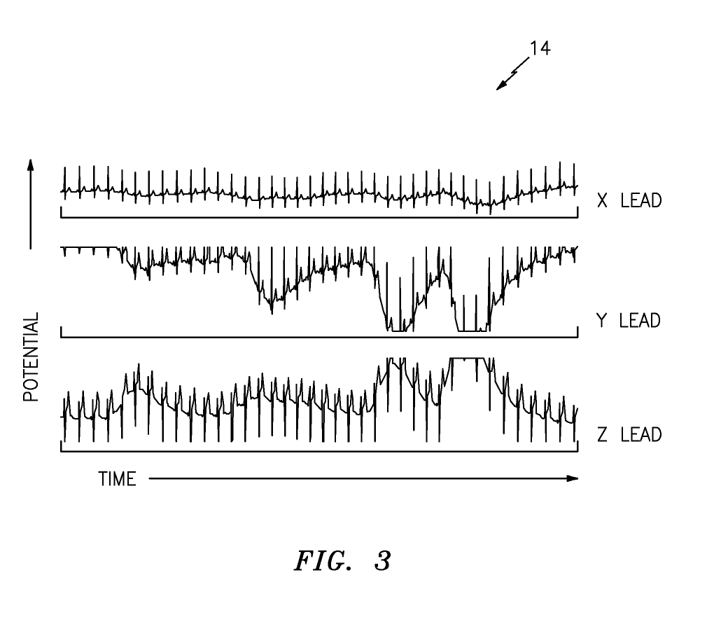

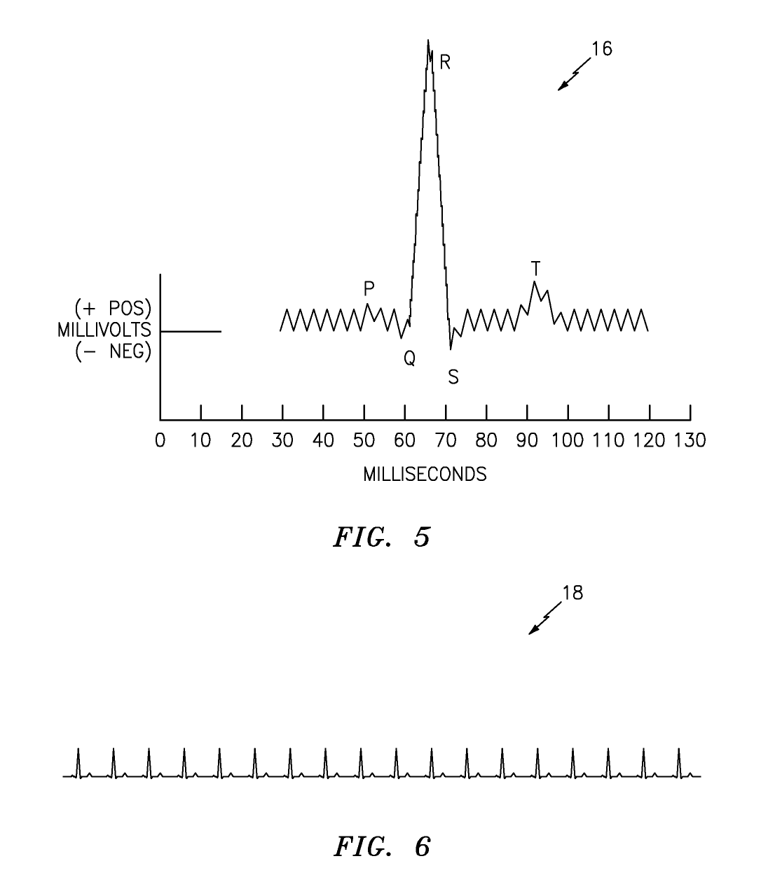

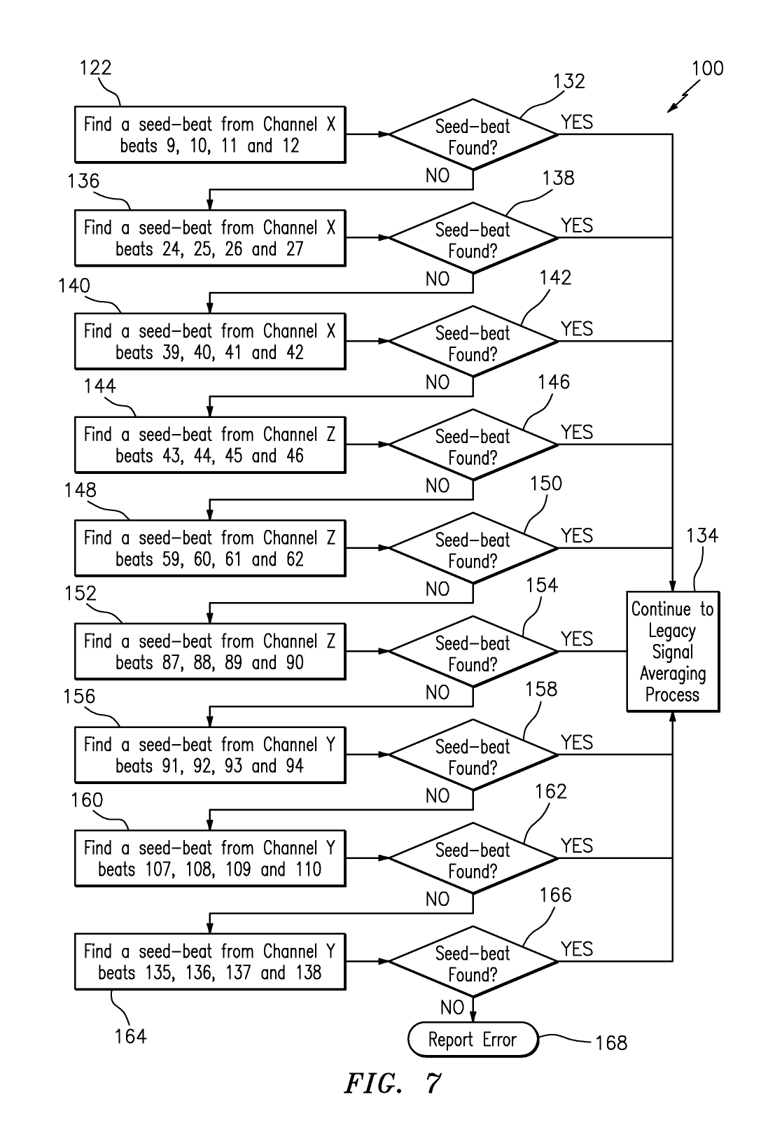

Applicants' FIG. 3 shows an example 14 of an electrocardiogram where a baseline shift is evident; and FIGS. 5 and 6 show the effect of electrical noise. Specifically, FIG. 5 depicts an exemplary view of a suboptimal heartbeat waveform 16 affected by noise; and, FIG. 6 depicts an exemplary view of an electrocardiogram time-series 18 with electrical noise.

Because signal averaging requires a number of consecutive beats that are similar in shape and position relative to the base line, such electrocardiograms, like those shown in FIGS. 3, 5 and 6, are considered suboptimal for SAECG purposes. Baseline drift and electrical noise tend to randomly change the shape of each heartbeat waveform. That random change increases the difficulty of accurately producing an average.

Usually electrocardiograms are affected by disturbances for periods of only several seconds during which time the patient may have moved or during some other momentary occurrence. Applicants' method can analyze automatically the heartbeat waveforms in any electrocardiogram, and then determine where to start signal averaging. The first heartbeat waveform to be used is called the “seed beat.”

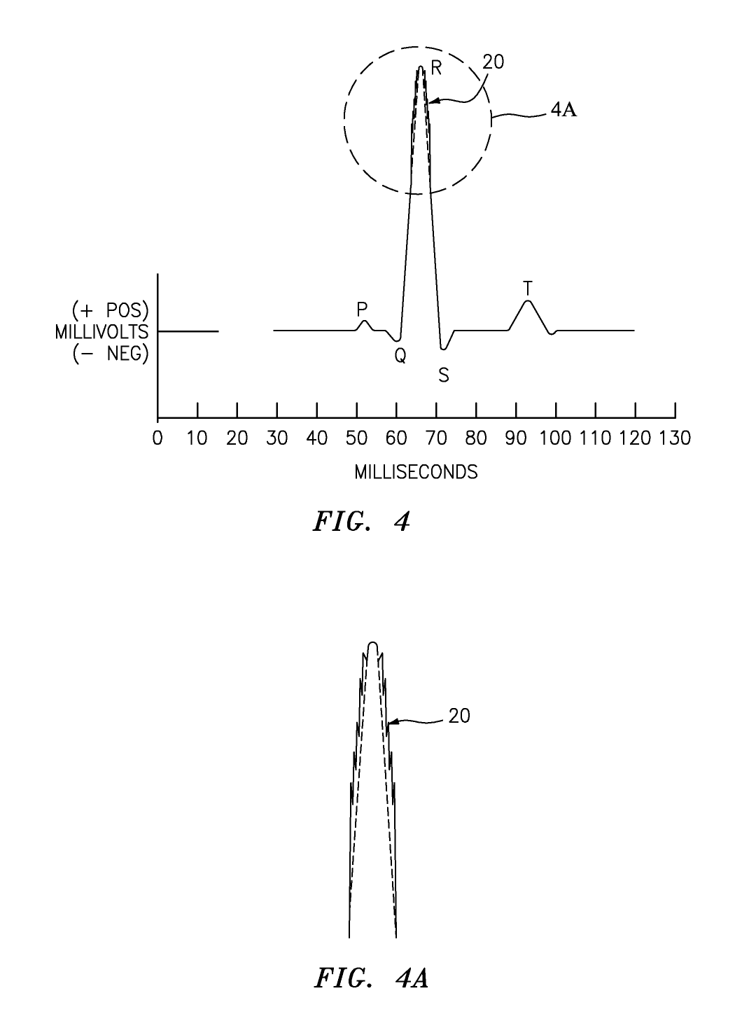

In previous systems, seed-beat selection could be handled manually or automatically. In manual mode, a medical practitioner could inspect the beats in a file and choose a suitable seed beat. In automatic mode, the seed beat is identified automatically in software. In either case, if the selected starting point is not followed by several similar heartbeat waveforms, then poor results or analytical failure can be experienced and the results are inconclusive. When a SAECG analysis is inconclusive, prediction of deadly arrhythmias by finding late micropotentials (e.g., 20 in FIGS. 4, 4A) is delayed and the patient may need to be recalled for further investigation. Therefore, starting the analysis at an analytically viable point in the electrocardiogram reduces the amount of time and effort required to complete an accurate diagnosis.

The inventive method of seed-beat selection is intended to be compatible with the prior art. Therefore, at the beginning of the selection process, the steps are identical to those of the prior art. If a suitable seed beat is found following the steps from the prior art, then the SAECG analysis procedure is performed on the remaining beats in the file and no further searching for seed beats needs to be performed. However, if a seed beat is not found using the procedures from the prior art, then the forward-looking inventive method is applied.

Applicants' first step in determining a seed beat (see FIG. 7) is to make a series of comparisons involving a sequence of four heartbeat waveforms (hereinafter “beat” or “beats”). The correlations can be performed in any number of ways including by polynomial fitting techniques, windowing, and comparison of coefficients after correcting for offset, or by performing a bitwise comparison of the graphic waveforms. (Refer to U.S. Pat. No. 5,609,158 to Chan at column 8, line 65 through column 9, line 20 for an exemplary description of the comparison of waveforms for similarity.)

In signal processing, cross-correlation is a measure of similarity of two waveforms as a function of a time-lag applied to one of them. This is also known as a sliding dot product or sliding inner-product. It is commonly used for searching a long-signal for a shorter, known feature. It also has applications in pattern recognition, single particle analysis, electron tomographic averaging, cryptanalysis, and neurophysiology.

For continuous functions, f and g, the cross-correlation is defined as:

$$( f * g ) ( t ) {=}^{def} {\int }_{- ∞}^{∞} {f}^{*} ( τ ) g ( t + τ ) d τ ,$$

where f * denotes the complex conjugate off This is “Equation 1.”

Similarly, for discrete functions, the cross-correlation is defined as:

$$( f * g ) [ n ] {=}^{def} {\sum }_{m = - ∞}^{∞} {f}^{*} [ m ] g [ n + m ] ,$$

where f * again denotes the complex conjugate off This is “Equation 2.”

Equation 2 (for discrete math functions) applies to digital signal processing. Applicants apply Equation 2 in their cross-correlations.

For one skilled in the art, it is clear that Equation 2 can be implemented by various computational means, e.g., a programming language like C/C++ compiled to run on a PC, or implemented in firmware for real-time signal processing by a microprocessor or DSP chip.

In any case, Applicants' preferred procedure requires an automated identification of a series of four consecutive heartbeats containing at least three out of four highly correlated (i.e., similar) waveforms as compared to one another. Applicants use a software driven process which determines whether the four beats deviate from one another.

A waveform morphology that differs from the others in the set by no more than 1% (i.e., cross-correlates within at least 99%) is considered a matching waveform.

Generally, the preferred process of comparing four sequential beats to find a seed beat follows a set of simple rules. For example, as depicted in FIG. 8, consider a series of sequential beats A, B, C, and D. The beats are cross-correlated (in other words, “compared”) with each other exhausting all possible combinations (i.e., comparisons are performed as follows: A to B, B to C, C to D, A to C, A to D, and finally B to D). The cross-correlations result in six true or false outcomes. If a pair of beats is cross-correlated to at least 99% (i.e., the beats deviate by less than 1%), then a score of 0 (zero) for affirmation (i.e., negative logic) is given to its respective cross-correlation. After the six cross-correlations are complete, the number of zero results is summed. Six zeros signify that all beats are the same, in which case the fourth beat in the sequence (beat “D”) is selected as the seed beat. Three zeros means there is one odd beat, and in that case, the third or fourth beat in the sequence is selected as the seed beat depending on further inspection of the cross-correlations. Any other number of zeros means that there are two beats of each type, or more than two different types. If there is a total of 1, 2, 4 or 5 zeros, then no beat can be selected as the seed beat.

By way of example, consider four consecutive heartbeat waveforms M, N, M, and M obtained from patient ECG data 101. The four waveforms are designated in respective order as the A, B, C, and D waveforms (FIG. 8 at 102). Comparing waveform A to B (i.e., “M” to “N”) (FIG. 8 at 104) the waveforms do not match so the result of the comparison yields a “1” which is added to an accumulating total (FIG. 8 at 106) starting with zero. After the first comparison then, the total logical score is 1. Now, comparing waveform B to C (i.e., “N” to “M”) (FIG. 8 at 110) again the waveforms do not match and again the comparison yields a result of “1” which is added to the accumulating total yielding a logical score of 2 (FIG. 8 at 112). Next, waveforms C and D are compared (i.e., “M” to “M”) (FIG. 8 at 114). The two waveforms match yielding a zero and resulting in an unchanged accumulated total score of 2. Next waveform A is compared to waveform C (i.e., “M” to “M”) (FIG. 8 at 116).

Since the two waveforms are cross-correlated at 99% (or better), the comparison results in a zero score so the accumulating total remains at 2. Next, waveforms A and D are compared (i.e., “M” to “M”) (FIG. 8 at 118) again yielding a zero result with an accumulated logical total of 2. Continuing, waveform B is compared with waveform D (i.e., “N” to “M”) (FIG. 8 at 120). Since B and D do not match the comparison yields a logical score of 1 which is added to the accumulating logical total incrementing its value to 3 (FIG. 8 at 122). To determine the number of zeros the accumulated total is subtracted from six (FIG. 8 at 124; six is the maximum possible number of zeros). In this example there are three zero results, and since both waveforms C and D match (FIG. 8 at 126; three zeros and A=B=C not true, requires C and D to match), the software would select beat D as the seed beat (FIG. 8 at 127).

If and only if the A and B, A and C, and B and C waveforms are all correlated yielding zero results, and the D beat is not correlated, then the third beat (beat C) is selected as the seed beat (FIG. 8 at 128). Otherwise beat D is selected. The overall process selects the D beat preferentially when both C and D beats are acceptable. If the number of zeros is 1, 2, 4, or 5, then the program repeats the process on another set of four beats or if it has reached the end of the file, it stops and reports an error (FIG. 8 at 129).

Applying the above steps (see Applicants' FIG. 7) to an electrocardiogram as implemented in the inventive method, the first four beats to be inspected are beats 9, 10, 11 and 12 (FIG. 7 at 132). The first eight beats are ignored because they often exhibit non-uniformity during measuring equipment stabilization. If the beats are sufficiently similar, either beat 11 or beat 12 is selected as a preliminary seed beat and the SAECG process continues from the seed beat (FIGS. 7 at 122 and 132); however, if no seed beat is found, the search goes on.

Continuing the process, beat 13 is skipped and four more beats including beats 24, 25, 26, and 27 are compared as described above and either beat 26 or beat 27 is chosen as the seed beat (FIGS. 7 at 136 and 138). The first set of four beats is not compared to the second set of four beats.

SAECG analysis is then performed on the next 100 to 300 beats in the file. The actual number of beats is dependent on when the signal-averaged noise level reaches a suitable threshold, as taught in the prior art. Applicants follow the Simson method disclosed in U.S. Pat. No. 4,422,459 to Simson, and claimed in his claim 10. Applicants' preferred threshold is a standard noise deviation of less than 0.3 uV.

If beats 24 through 27 are not well correlated, then, in the case of the prior art method, the process stops and an error message alerts the operator that the file cannot be analyzed; however, in the inventive method, software continues to analyze certain remaining heartbeat waveforms in the ECG searching for a viable seed beat.

If no viable seed beat is found by beats 24, 25, 26 and 27, additional searching can occur. For example, up to seven more sets of heartbeats (as represented by their electrocardiographic waveforms) can be searched from Channels X, Y and Z. (See FIG. 7, which shows sample additional search steps at 138, 140, 142, 144, 146, 148, 150, 152, 154, 156, 158, 160, 162, 164 and 166). Those additional steps refer to the following sets of heartbeats: beats 39, 40, 41 and 42 from Channel X; beats 43, 44, 45 and 46 from Channel Z; beats 59, 60, 61 and 62 from Channel Z; beats 87, 88, 89 and 90 from Channel Z; beats 91, 92, 93 and 94 from Channel Y; beats 107, 108, 109 and 110 from Channel Y; and beats 135, 136, 137 and 138 from Channel Y.

In essence, the process looks forward through the file (i.e., a series of many sets of consecutive heartbeats) to try to find a point in the file where the heartbeat waveforms have stabilized, and if stabilization has occurred, to see if there is data in the remainder of the file that can be analyzed by SAECG. While all searching thus far described has been in channel X, the inventive process also looks at channel Y and channel Z for useable data.

If no suitable seed beats are found in any channel, then the method alerts the operator that an error has occurred and that the file is unsuitable for SAECG analysis (FIG. 7 at 168).

Applicants' process employs a rule-based expert system to overcome challenging obstacles presented by suboptimal HI-RES electrocardiograms. This process has been implemented in Arrhythmia Research Technology, Inc.'s, PREDICTOR® software running on Windows OS, and tested on one-hundred-ten HI-RES ECG data files that were acquired at a sampling rate of 1 kHz per channel from the orthogonal X, Y, Z leads of supine patients and volunteer subjects using a Nihon Kohden ECG 1500-series electrocardiograph.

Applicants' test results were compared to the decision making process of a human expert. The comparison demonstrated a significant improvement (from 53% to 98%) in the yield of HI-RES ECG records that automatically completed the signal averaging process to form the final SAECG vector magnitude result.

The process leaves previously successful template formation processes unchanged. It automatically recognizes suboptimal sections of HI-RES ECG files and applies “expert system” decision rules to automatically search the HI-RES ECG files to identify stable, quiescent QRS complexes. It then continues to accept incoming QRS complexes at the 99% cross-correlation percentile.

Applicants' testing successfully demonstrated their approach to using Artificial

Intelligence (“Al”) techniques to automatically form template beats when applied to previously problematic HI-RES ECG time-series that could not be signal-averaged automatically (e.g., FIG. 3). The software run-time adds no significant computational delay to the signal averaging process and has the potential for increasing productivity gains in clinical workflow and patient/clinician satisfaction. It can also save lives by speeding the discovery of dangerous arrhythmias.

Applicants' preferred method can be thought of broadly as comprising the following sequential steps:

a. analyzing electrocardiographic waveforms of sets of four consecutive heartbeats of a person, located along preordained positions in the X, Y, and Z channels of a digital electrocardiogram, to determine the first time, if any, a set of four consecutive heartbeats is represented by four consecutive waveforms in which at least three out of the four consecutive waveforms deviate from each other by less than a preselected threshold amount or percentage (preferably 1%); and

b. upon determining a set of four consecutive heartbeats is represented by a set of four consecutive waveforms in which at least three out of the four consecutive waveforms deviate from each other by less than the preselected percentage:
i. identifying heartbeat four, in the set of four consecutive heartbeats, as a seed beat for signal-averaged electrocardiography if all four consecutive waveforms deviate from each other by less than the preselected percentage; and
ii. identifying heartbeat three, in the set of four consecutive heartbeats, as a seed beat for signal-averaged electrocardiography if only the first three of the four consecutive waveforms, in the set of four consecutive waveforms, deviate from each other by less than the preselected percentage.

Additional steps can include selecting heartbeat three, rather than heartbeat four, where:
a. identifying heartbeat four, in the set of four consecutive heartbeats, as a seed beat for signal-averaged electrocardiography if only a first, second and fourth waveform, in the set of four consecutive waveforms, deviate from each other by less than the preselected percentage.

Applicants recognize that the preferred cross-correlation threshold (i.e., within at least 99%) may be too high for difficult SAECG data sets, or when using their method for

P-wave instead of R-wave SAECG. For those instances, Applicants reduce their cross-correlation threshold to at least 95%. In other words, the two compared beats deviate from each other by less than 5%.

It should be understood by those skilled in the art that obvious modifications can be made to Applicants' preferred method without departing from the spirit of the invention. For example, sets of five beats, instead of four beats, could be analyzed to pick the optimal seed beat for SAECG.

It should be further understood by those skilled in the art that the choices of location and number of attempts for selecting seed beats, as disclosed here, represent design decisions in a particular embodiment, and should not be interpreted as limiting on the instant invention. For example, the invention includes selection of successive sets of four or more heartbeats without resorting to interspersing one or more beats to be skipped. Further, the instant invention equally applies to the selection of at least four consecutive waveforms in each of the X, Y and/or Z channels, without necessarily moving to a different location in the data file for the different channels.

Accordingly, primary reference should be made to the accompanying claims rather than the foregoing Specification to determine the scope of the invention.

We claim:

1. A method comprising:
a. analyzing electrocardiographic waveforms of sets of four consecutive heartbeats of a person, located along preordained positions in X, Y, and Z channels of a digital electrocardiogram, to determine the first time, if any, a set of four consecutive heartbeats is represented by four consecutive waveforms in which at least three out of the four consecutive waveforms deviate from each other by less than 1%; and
b. upon determining a set of four consecutive heartbeats is represented by a set of four consecutive waveforms in which at least three out of the four consecutive waveforms deviate from each other by less than 1%:
i. identifying heartbeat four, in the set of four consecutive heartbeats, as a seed beat for signal-averaged electrocardiography if all four consecutive waveforms deviate from each other by less than 1%; and
ii. identifying heartbeat three, in the set of four consecutive heartbeats, as a seed beat for signal-averaged electrocardiography if only a first three of the four consecutive waveforms, in the set of four consecutive heartbeats, deviate from each other by less than 1%.

2. The method of claim 1 further comprising:
a. upon determining a set of four consecutive heartbeats is represented by a set of four consecutive waveforms in which at least three out of the four consecutive waveforms deviate from each other by less than 1%:
i. identifying heartbeat four, in the set of four consecutive heartbeats, as a seed beat for signal-averaged electrocardiography if only a first, second and fourth waveforms, in the set of four consecutive waveforms, deviate from each other by less than 1%.

3. The method of claim 2 further comprising:
a. terminating the analysis and producing an error message if:
i. none of the sets analyzed is represented by four consecutive waveforms, which deviate from each other by less than 1%; and
ii. none of the additional sets analyzed has at least three waveforms which deviate from each other by less than 1%.

4. The method of claim 3 further comprising: whereby an appropriate seed beat can be selected despite suboptimal electrocardiograms.

5. A method comprising:
a. automatically analyzing electrocardiographic waveforms of consecutive sets of four heartbeats, of a person, located along preordained positions in an X channel, then a Z channel, and then a Y channel of a high-resolution electrocardiogram, to determine the first set of four heartbeats represented by at least three out of four waveforms which deviate from each other by less than a preselected percentage and thereafter:
i. identifying heartbeat three of the first set to serve as a seed beat to perform signal-averaged electrocardiogram analysis if the first three consecutive waveforms deviate from each other by less than the preselected percentage;
ii. identifying heartbeat four of the first set to serve as a seed beat to perform signal-averaged electrocardiogram analysis upon finding only the first, second, and fourth waveforms, of the four waveforms representing the first set, deviate from each other by less than the preselected percentage.

6. The method of claim 5 wherein the preselected percentage is 5%.

7. The method of claim 5 wherein the preselected percentage is 1%.

8. The method of claim 5 further comprising:
a. upon determining a set of four consecutive heartbeats is represented by a set of four consecutive waveforms in which at least three out of the four consecutive waveforms deviate from each other by less than the preselected percentage:
i. identifying heartbeat four, in the set of four consecutive heartbeats, as a seed beat for signal-averaged electrocardiography if only a first, second and fourth waveform, of the four waveforms representing the first set, deviate from each other by less than the preselected percentage.

9. The method of claim 8 further comprising: whereby a seed beat can be selected despite a suboptimal electrocardiogram.

10. A method comprising:
a. analyzing electrocardiographic waveforms of sets of four consecutive heartbeats of a person, located along preordained positions in X, Y, and Z channels of a digital electrocardiogram, to determine the first time, if any, a set of four consecutive heartbeats is represented by four consecutive waveforms in which at least three out of the four consecutive waveforms correlate to each other by at least 95%; and
b. upon determining a set of four consecutive heartbeats is represented by a set of four consecutive waveforms in which at least three out of the four consecutive waveforms correlate to each other by at least 95%:
i. identifying heartbeat four, in the set of four consecutive heartbeats, as a seed beat for signal-averaged electrocardiography if all four consecutive waveforms correlate to each other by at least 95%; and
ii. identifying heartbeat three, in the set of four consecutive heartbeats, as a seed beat for signal-averaged electrocardiography if only a first three of the four consecutive waveforms, in the set of four consecutive heartbeats, correlate to each other by at least 95%.

11. The method of claim 10 further comprising:
a. upon determining a set of four consecutive heartbeats is represented by a set of four consecutive waveforms in which at least three out of the four consecutive waveforms correlate to each other by at least 95%:
i. identifying heartbeat four, in the set of four consecutive heartbeats, as a seed beat for signal-averaged electrocardiography if only a first, second and fourth waveforms, in the set of four consecutive waveforms, correlate to each other by at least 95%.

12. The method of claim 11 further comprising:
a. terminating the analysis and producing an error message if:
i. none of the sets analyzed is represented by four consecutive waveforms, which correlate with each other within at least 95%; and
ii. none of the additional sets analyzed has at least three waveforms which correlate to each other by at least 95%.

13. The method of claim 12 further comprising: whereby an appropriate seed beat can be selected despite suboptimal electrocardiograms.

14. A method comprising:
a. automatically analyzing electrocardiographic waveforms of consecutive sets of at least four heartbeats, of a person, located along preordained positions in X, Z, Y channels of an electrocardiogram, to determine the first time a set of at least four heartbeats is represented by consecutive waveforms in which at least three waveforms deviate from each other by less than 5%; and
b. identifying thereafter a heartbeat, in the first set, to serve as a seed beat to perform signal-averaged electrocardiogram.

15. A method comprising:
a. automatically analyzing electrocardiographic waveforms of consecutive sets of at least four heartbeats, of a person, located along preordained positions in X, Z, Y channels of an electrocardiogram, to determine the first time a set of at least four heartbeats is represented by at least four consecutive waveforms which deviate from each other by less than 5%; and
b. identifying thereafter a heartbeat, in the set, to serve as a seed beat to perform signal-averaged electrocardiogram.
